(19) United States
(12) Patent Application Publication
(10) Pub. No.: US 2026/0000913 A1
(43) Pub. Date: Jan. 1, 2026

(54) APPARATUS AND METHOD OF IMAGE DEFORMATION BASED ON IMAGING UNCERTAINTY

(71) Applicant: Accuray LLC, Madison, WI (US)

(72) Inventors: Philip Corrado, Winston-Salem, NC (US); Calvin R. Maurer, JR., San Jose, CA (US); Richard Holloway, Chapel Hill, NC (US); Jari Toivanen, Redwood City, CA (US); Charles Brandon Frederick, Raleigh, NC (US)

(21) Appl. No.: 18/759,738

(22) Filed: Jun. 28, 2024

(51) Int. Cl. A61N 5/10; G06T 7/00; G06T 7/73; G06T 11/00

(52) U.S. Cl. CPC A61N 5/1039; A61N 5/1071; G06T 7/0016; G06T 7/74; G06T 11/008; G06T 2200/04; G06T 2207/10081; G06T 2207/20101; G06T 2207/30004; G06T 2211/448

(57) ABSTRACT

An apparatus includes a memory to store first volumetric image data representing a first volumetric image of an anatomical region having a volume of interest (VOI). The first volumetric image includes several first voxels at first locations in the first volumetric image. The first volumetric image data includes first uncertainty values corresponding to the several first voxels. The first uncertainty values represent probabilities that the corresponding plurality of first voxels are part of the VOI. The memory is to store second volumetric image data representing a second volumetric image of the anatomical region. The apparatus includes a processing device operatively coupled to the memory. The processing device is to determine a deformation field to map the several first voxels to second locations in the second volumetric image. The deformation field is based in part on the first uncertainty values.

### TECHNICAL FIELD

The present disclosure relates to radiation therapy systems. More particularly, the present disclosure related to radiation therapy systems for planning or delivering radiation therapy to an anatomical region.

### BACKGROUND

In radiation treatment, a radiation therapy system may plan, deliver, or analyze radiation treatment of a volume of interest, such as a tumor or an organ at risk, in an anatomical region of a patient. Localization of the volume of interest can be annotated manually, computed automatically, or generated in a semi-automated fashion.

### BRIEF DESCRIPTION OF THE DRAWINGS

The present disclosure will be understood more fully from the detailed description given below and from the accompanying drawings of various implementations of the disclosure.

FIG. 1 illustrates a helical radiation delivery system, in accordance with embodiments described herein.

FIG. 2 illustrates a robotic radiation treatment system, in accordance with embodiments described herein.

FIG. 3 illustrates a C-arm gantry-based radiation treatment system, in accordance with embodiments described herein.

FIG. 4 illustrates a flow diagram of a method of generating beam control data based on uncertainty values, in accordance with embodiments described herein.

FIG. 5 illustrates a volumetric image of an anatomical region having a volume of interest, in accordance with embodiments described herein.

FIG. 6 illustrates voxels of a volumetric image having associated uncertainty values, in accordance with embodiments described herein.

FIG. 7 illustrates a radiation delivery system, in accordance with embodiments described herein.

FIG. 8 illustrates a segmentation map representing a volume of interest, in accordance with embodiments described herein.

FIG. 9 illustrates a dose-volume histogram for a volume of interest, in accordance with embodiments described herein.

FIG. 10 illustrates a flow diagram of a method of determining a deformation field, in accordance with embodiments described herein.

FIG. 11 illustrates volumetric images mapped to each other by a deformation field, in accordance with embodiments described herein.

FIG. 12 illustrates a deformed image having a contour, in accordance with embodiments described herein.

FIG. 13 illustrates a deformed image having an uncertain region, in accordance with embodiments described herein.

FIG. 14 illustrates a flow diagram of a method of determining a dose accumulation based on uncertainty values, in accordance with embodiments described herein.

FIG. 15 illustrates a block diagram of an example computing device that may perform one or more of the operations described herein, in accordance with embodiments described herein.

### DETAILED DESCRIPTION

Described herein are embodiments for using uncertainty values associated with voxels of a volumetric image, taken of a volume of interest (VOI), to generate beam control data to direct a treatment beam relative to the VOI. The uncertainty values can represent probabilities that the voxels belong to the VOI.

Existing radiation therapy systems deliver a radiation beam to a target, such as a cancerous tumor, to treat disease. A treatment plan is commonly developed, based on an image of a patient, which includes outlines of the target. The radiation therapy system runs software to determine, based on the outlines, how to control the radiation beam.

A drawback of existing treatment planning based on outlines developed from patient images exists in the inability to take uncertainty of the target location into account. Uncertainty stems from several sources, including: limitations in the image acquisition/reconstruction process, limited image contrast between organs, and preferences/tendencies of an annotator or algorithm designer in subjective areas (which can cause interobserver variability). For example, there may exist in the mind of a human annotator or in an intermediate step of an algorithm a probability mapping assigning each voxel of the image to a target. The assignment may be made with a certain confidence, however, at a target boundary or border, the user or algorithm may be unsure about whether a particular voxel is part of the target. Nonetheless, the radiation therapy systems have volumetric imagers to produce the images (e.g., x-ray images) of the target, and the annotator must evaluate and determine whether voxels of the image are inside of outside of the target. Typically, voxels having certain characteristics, e.g., an intensity above a threshold, are considered to be inside of the target and voxels lacking the characteristic, e.g., having an intensity below the threshold, are considered to be outside of the target. An outline, e.g., a border, is displayed between the differentiated voxels. This binary determination, which deems a voxel as in or out of the target fails to recognize or account for uncertainty in the image. More particularly, there is some uncertainty in whether voxels are in or out of the target based on a thresholded character (with the uncertainty typically increasing toward the border) but that uncertainty is typically not accounted for in treatment plans. As a result, treatment plans can direct radiation to tissue that is thresholded in, but is actually not part of the target, and may not direct radiation to tissue that is thresholded out, but is actually part of the target.

Aspects of the disclosure may remedy the above and other deficiencies by having a radiation therapy system that accounts for uncertainty in imaged anatomy to more accurately deliver therapy. The radiation therapy system can, rather than thresholding voxels in a binary manner to determine whether the voxels are inside or outside of a volume of interest, utilize uncertainty information to improve the radiation therapy process. This concept, of using uncertainty associated with voxels to contribute to therapy decisions and/or treatment, can be applied in treatment planning, organ tracking, image registration, or dose accumulation, as described below.

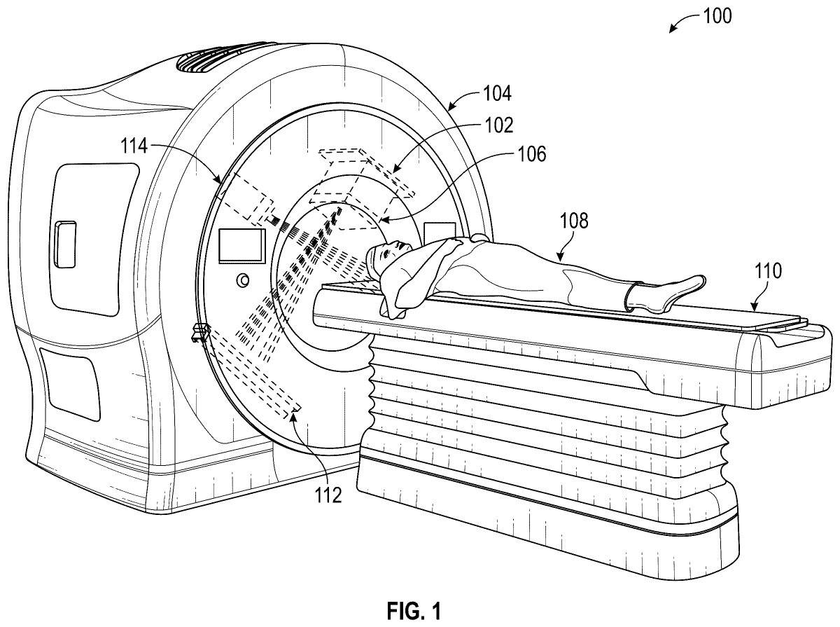

Referring to FIG. 1, a helical radiation delivery system is shown in accordance with embodiments described herein. An apparatus 100 may include a helical radiation delivery system. The helical delivery system may include a linear accelerator (LINAC) 102 mounted to a ring gantry 104. The LINAC 102 may be used to generate a radiation beam (i.e., treatment beam) by directing an electron beam towards a target made of material with high atomic number (Z) to produce x-rays. The treatment beam may deliver radiation to, or relative to, a target region (e.g., a volume of interest (VOI) such as a tumor or an organ at risk). The treatment system further includes a multileaf collimator (MLC) 106 coupled with the distal end of the LINAC 102. The MLC includes a housing that houses multiple leaves that are movable to adjust an aperture of the MLC to enable shaping of the treatment beam. In embodiments, the MLC 106 may be a binary MLC that includes a plurality of leaves arranged in two opposing banks, where the leaves of the two opposing banks are interdigitated with one another and can be opened or closed to form an aperture. In some embodiments, the MLC 106 may be an electromagnetically actuated MLC. In embodiments, MLC 106 may be any other type of MLC. The ring gantry 104 has a toroidal shape in which the patient 108 extends through a bore of the ring/toroid and the LINAC 102 is mounted on the perimeter of the ring and rotates about the axis passing through the center to irradiate a target region with beams delivered from one or more angles around the patient. During treatment, a patient 108 may be simultaneously moved through the bore of the gantry on a treatment couch 110.

The helical radiation delivery system 100 includes an imaging system 114, comprising the LINAC 102 as an imaging source and an x-ray detector 112. The LINAC 102 may be used to generate a mega-voltage x-ray image (MVCT) of a VOI of patient 108 by directing a sequence of x-ray beams at the ROI which are incident on the x-ray detector 112 opposite the LINAC 102 to image the patient 108 for setup and generate pre-treatment images. In one embodiment, the helical radiation delivery system 100 may also include a secondary imaging system 114 having, for example, a kV imaging source. The imaging source may be mounted orthogonally relative to the LINAC 102 (e.g., separated by 90 degrees) on the ring gantry 104 and may be aligned to project an imaging x-ray beam at the VOI and to illuminate an imaging plane of a detector after passing through the patient 108.

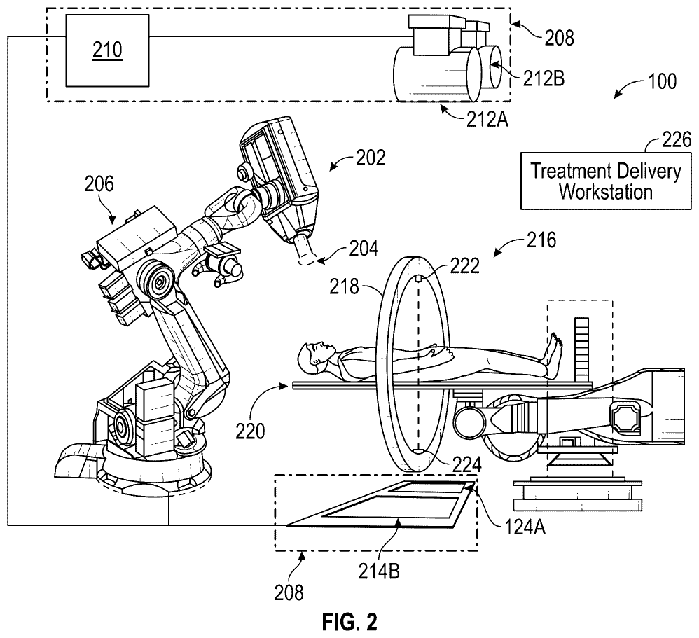

Referring to FIG. 2, a robotic radiation treatment system is shown in accordance with embodiments described herein. The apparatus 100 can include a radiation treatment system having a linear accelerator (LINAC) 202 that acts as a radiation treatment source and an MLC 204 coupled with the distal end of the LINAC 202 to shape the treatment beam. In one embodiment, the LINAC 202 is mounted on the end of a robotic arm 206 having multiple (e.g., 5 or more) degrees of freedom in order to position the LINAC 202 to irradiate a pathological anatomy (e.g., target) with beams delivered from many angles, in many planes, in an operating volume around a patient. Treatment may involve beam paths with a single isocenter, multiple isocenters, or with a non-isocentric approach.

LINAC 202 may be positioned at multiple different nodes (predefined positions at which the LINAC 202 is stopped and radiation may be delivered) during treatment by moving the robotic arm 206. At the nodes, the LINAC 202 can deliver one or more radiation treatment beams to a target, where the radiation beam shape is determined by the leaf positions in the MLC 204. The nodes may be arranged in an approximately spherical distribution about a patient. The particular number of nodes and the number of treatment beams applied at each node may vary as a function of the location and type of pathological anatomy to be treated.

In another embodiment, the robotic arm 206 and LINAC 202 at its end may be in continuous motion between nodes while radiation is being delivered. The radiation beam shape and 2-D intensity map is determined by rapid motion of the leaves in the MLC 204 during the continuous motion of the LINAC 202.

In some embodiments, the radiation treatment system may include an imaging system 208 having a processing device 210 connected with x-ray sources 212A and 212B (i.e., imaging sources) and fixed x-ray detectors 214A and 214B. The imaging system 208 may be utilized to generate additional imaging beams. Alternatively, the x-ray sources 212A, 212B and/or x-ray detectors 214A, 214B may be mobile, in which case they may be repositioned to maintain alignment with the target, or alternatively to image the target from different orientations or to acquire many x-ray images and reconstruct a three-dimensional (3D) cone-beam CT. In one embodiment, LINAC 202 serves as an imaging source, where the LINAC power level is reduced to acceptable levels for imaging.

Imaging system 208 may perform computed tomography (CT) such as cone beam CT or helical megavoltage computed tomography (MVCT), and images generated by imaging system 208 may be two-dimensional (2D) or three-dimensional (3D). The two x-ray sources 212A and 212B may be mounted in fixed positions on the ceiling of an operating room and may be aligned to project x-ray imaging beams from two different angular positions (e.g., separated by 90 degrees) to intersect at a machine isocenter (referred to herein as a treatment center, which provides a reference point for positioning the patient on a treatment couch 220 during treatment) and to illuminate imaging planes of respective detectors 214A and 214B after passing through the patient. In one embodiment, imaging system 208 provides stereoscopic imaging of a target and the surrounding volume of interest (VOI). In other embodiments, imaging system 208 may include more or less than two x-ray sources and more or less than two detectors, and any of the detectors may be movable rather than fixed. In yet other embodiments, the positions of the x-ray sources and the detectors may be interchanged. Detectors 214A and 214B may be fabricated from a scintillating material that converts the x-rays to visible light (e.g., amorphous silicon), and an array of CMOS (complementary metal oxide silicon) or CCD (charge-coupled device) imaging cells that convert the light to a digital image that can be compared with a reference image during an image registration process that transforms a coordinate system of the digital image to a coordinate system of the reference image, as is well known to the skilled artisan. The reference image may be, for example, a digitally reconstructed radiograph (DRR), which is a virtual x-ray image that is generated from a 3D CT image based on simulating the x-ray image formation process by casting rays through the CT image.

In one embodiment, IGRT delivery system also includes a secondary imaging system 216. Imaging system 216 may be a Cone Beam Computed Tomography (CBCT) imaging system. Alternatively, other types of volumetric imaging systems may be used. The secondary imaging system 216 includes a rotatable gantry 218 (e.g., a ring) attached to an arm and rail system (not shown) that move the rotatable gantry 218 along one or more axes (e.g., along an axis that extends from a head to a foot of the treatment couch 220. An imaging source 222 and a detector 224 are mounted to the rotatable gantry 218. The rotatable gantry 218 may rotate 360 degrees about the axis that extends from the head to the foot of the treatment couch. Accordingly, the imaging source 222 and detector 224 may be positioned at numerous different angles. In one embodiment, the imaging source 222 is an x-ray source and the detector 224 is an x-ray detector. In one embodiment, the secondary imaging system 216 includes two rings that are separately rotatable. The imaging source 222 may be mounted to a first ring and the detector 224 may be mounted to a second ring. In one embodiment, the rotatable gantry 218 rests at a foot of the treatment couch during radiation treatment delivery to avoid collisions with the robotic arm 206.

The image-guided radiation treatment system may further be associated with a treatment delivery workstation 226. The treatment delivery workstation may be remotely located from the radiation treatment system in a different room than the treatment room in which the radiation treatment system and patient are located. The treatment delivery workstation 226 may include a processing device (which may be processing device 210 or another processing device) and memory that modify a treatment delivery to the patient based on uncertainty in one or more images, as described herein.

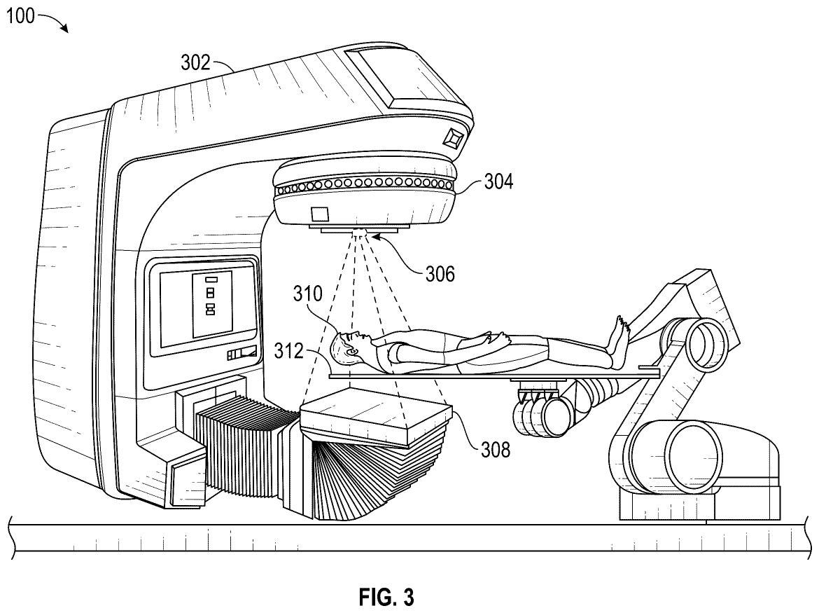

Referring to FIG. 3, a C-arm gantry-based radiation treatment system is shown in accordance with embodiments described herein. The apparatus 100 can include a C-arm system. The C-arm system allows the beam energy of a LINAC to be adjusted during treatment and may allow the LINAC to be used for both x-ray imaging and radiation treatment. In another embodiment, the system may include an onboard kV imaging system to generate x-ray images and a separate LINAC to generate the higher energy therapeutic radiation beams. The system includes a C-arm gantry 302, a LINAC 304, an MLC 306 coupled with the distal end of the LINAC 304 to shape the beam, and a portal imaging detector 308. The C-arm gantry 302 may be rotated to an angle corresponding to a selected projection and used to acquire an x-ray image of a VOI of a patient 310 on a treatment couch 312. In embodiments that include a portal imaging system, the LINAC 304 may generate an x-ray beam that passes through the target of the patient 310 and are incident on the portal imaging detector 308, creating an x-ray image of the target. After the x-ray image of the target has been generated, the beam energy of the LINAC 304 may be increased so the LINAC 304 may generate a radiation beam to treat a target region of the patient 310. In another embodiment, the kV imaging system may generate an x-ray beam that passes through the target of the patient 310, creating an x-ray image of the target. In some embodiments, the portal imaging system may acquire portal images during the delivery of a treatment. The portal imaging detector 308 may measure the exit radiation fluence after the beam passes through the patient 310. This may enable internal or external fiducials or pieces of anatomy (e.g., a tumor or bone) to be localized within the portal images.

Alternatively, the kV imaging source or portal imager and methods of operations described herein may be used with yet other types of gantry-based systems. In some gantry-based systems, the gantry rotates the kV imaging source and LINAC around an axis passing through the isocenter. Gantry-based systems include ring gantries having generally toroidal shapes in which the patient's body extends through the bore of the ring/toroid, and the kV imaging source and LINAC are mounted on the perimeter of the ring and rotates about the axis passing through the isocenter. Gantry-based systems may further include C-arm gantries, in which the kV imaging source and LINAC are mounted, in a cantilever-like manner, over and rotates about the axis passing through the isocenter. In another embodiment, the kV imaging source and LINAC may be used in a robotic arm-based system, which includes a robotic arm to which the kV imaging source and LINAC are mounted as discussed above. Aspects of the present disclosure may further be used in other such systems such as a gantry-based LINAC system, static imaging systems associated with radiation therapy and radiosurgery, proton therapy systems using an integrated image guidance, interventional radiology, and intraoperative x-ray imaging systems, etc.

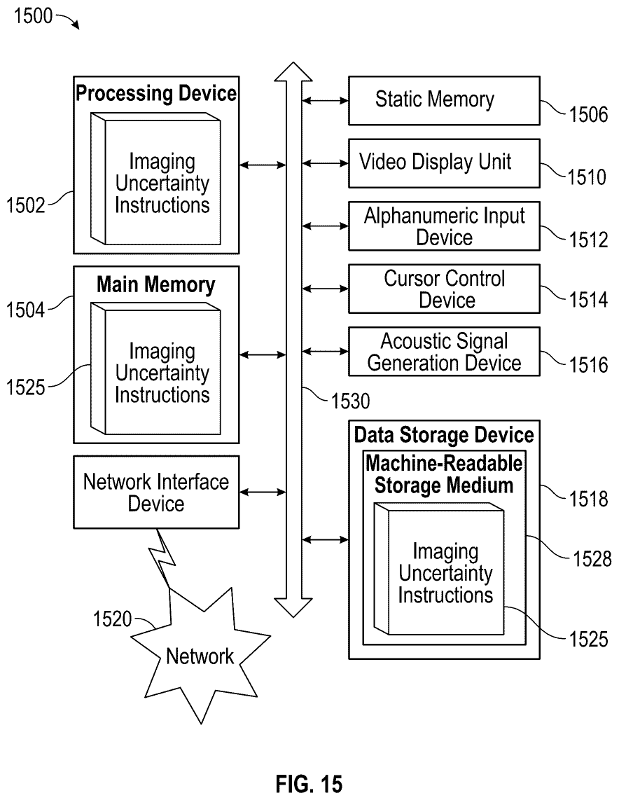

The apparatus 100 can be used to plan, deliver, or analyze radiation therapy, and accordingly, may be referred to as a radiation therapy system. The radiation therapy system can include a computing device (FIG. 15) to control the system components. For example, the apparatus 100 can include a memory to store data, as described below, and a processing device operatively coupled to the memory to perform operations of the methods described herein. More particularly, the processing device can execute instructions stored on a non-transitory computer-readable storage medium to cause the apparatus 100 to perform the operations, which are described in more detail below.

Treatment Planning and/or Organ Tracking Using Uncertainty

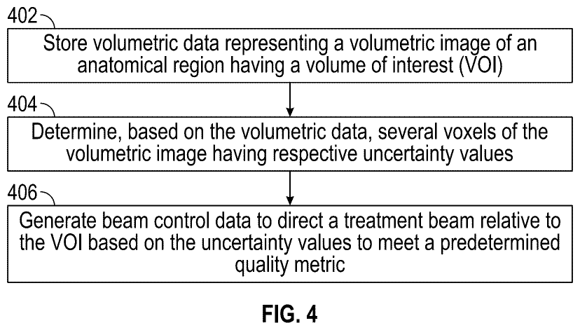

Referring to FIG. 4, a flow diagram of a method of generating beam control data based on uncertainty values is shown in accordance with embodiments described herein. The method illustrated in FIG. 4 can be an overarching method having operations that are used in treatment planning (e.g., prior to delivering a radiation beam to a patient) or in organ tracking (e.g., after and/or during delivery of the radiation beam to the patient). Furthermore, the operations can be understood with reference to FIGS. 5-9 and, thus, FIGS. 4-9 are alternately referred to below.

Referring to FIG. 5, a volumetric image of an anatomical region having a volume of interest is shown in accordance with embodiments described herein. At operation 402, volumetric data representing a volumetric image 502 of an anatomical region 504 having a VOI 506 can be stored in the memory of the apparatus 100. The anatomical region 504 captured in the volumetric image 502 may include, for example, a target 508 or an organ at risk (OAR) 509. For example, the volumetric image 502 can be an image of a thoracic region of a patient, and can include a target 508 that may be a portion of a lung having a cancerous tumor. The OAR 509 in the image may be a heart located adjacent to the tumor. The VOI 506 of the anatomical region 504 can be one or both of the target 508 or the OAR 509. For example, the target 508 may be a first VOI and the OAR 509 may be a second VOI. For ease of understanding, the VOI 506 in the anatomical region 504 is shown as discrete elliptical areas separated in space, however, it will be appreciated that the VOIs may be any shape in the image (for example, lung- or heart-shaped), and may be immediately adjacent or overlapping each other in the image.

At operation 404, the processing device of the apparatus 100 can determine, based on the volumetric data, several voxels 510 of the volumetric image 502. The imaging system of the apparatus 100 can capture one or more images of the anatomical region 504 to generate the volumetric data, and the processing device may use reconstruction techniques to generate the volumetric image 502. The volumetric image 502 can include several voxels 510. Only a few voxels 510 are shown in FIG. 5, but it will be appreciated by one skilled in the art that the VOI 506 can be segmented into a multitude of voxels 510 interlinked to define the anatomical structure in a virtual space.

In an embodiment, the processing device determines an uncertainty value associated with each of the voxels 510. More particularly, each voxel 510 can have a respective uncertainty value representing a probability that the voxel 510 belongs to the VOI 506. An estimate of the uncertainty value may be based on several factors, including: typical organ shape, image intensity, and imaging/reconstruction uncertainty.

Uncertainty based on typical organ shape may include a comparison between the shape of the OAR 509 in the volumetric image 502 and an expected shape of the OAR 509. For example, previous images of a tumor or an organ may be taken and compared to the volumetric image 502. The boundary of the VOI 506 can be determined from the comparison, and a proximity of the voxel 510 to the determined boundary may be directly correlated to an uncertainty that the voxel 510 is part of the VOI 506. More particularly, voxels 510 that are nearer to the boundary of the expected shape can have a lower probability of being part of the VOI 506.

Uncertainty based on image intensity may include a comparison of an intensity of the voxel 510 to a predetermined intensity threshold. More particularly, a user may set an intensity threshold for voxels 510 that are more likely to be within the VOI 506. When the voxel 510 meets the threshold, then it is more likely that the voxel 510 is part of the VOI 506.

Imaging/reconstruction uncertainty in the volumetric data can reflect imaging artifacts of the imaging system, which may be known and accounted for by the processing device. For example, the typical artifacts that arise during the imaging process include patient-based artifacts, physics-based artifacts, and hardware-based artifacts. Patient-based artifacts can include: motion artifacts, transient interruption of contrast, clothing artifacts, and jewelry artifacts. Physics-based artifacts can include: beam hardening, cupping artifacts, streak and dark bands, metal artifacts, high-density foreign material artifacts, partial volume averaging, quantum mottle (noise), photon starvation, aliasing, and truncation artifacts. Hardware-based artifacts can include: ring artifacts, tube arcing, out of field artifacts, air bubble artifacts, helical and multichannel artifacts, windmill artifacts, cone beam effect, multiplanar reconstruction (MPR) artifacts, zebra artifacts, and stair step artifacts. The reconstruction techniques used by the apparatus 100 to generate the volumetric image 502 may aim to reduce such artifacts. Such artifacts are known in the art and are not described in detail here in the interest of brevity. Notably, however, such artifacts are a contributing factor to uncertainty in whether voxels 510 belong to the VOI 506 and can contribute to a determination of whether voxels 510 are part of the VOI. By way of example, photon starvation is an artifact that is measurable in voxels of images taken by the imaging system, and can contribute to a determination of uncertainty associated with the voxels. Accordingly, the apparatus 100 can advantageously utilize knowledge of artifacts originating with the imaging system to determine uncertainty associated with voxels 510 in the volumetric image 502. More particularly, the imaging system, when generating the images and/or volumetric data, can include an additional channel of information for estimating uncertainty. Such determination may not be possible, for example, when images are captured by a different apparatus 100, and there is no knowledge of the system parameters that were used to capture the images.

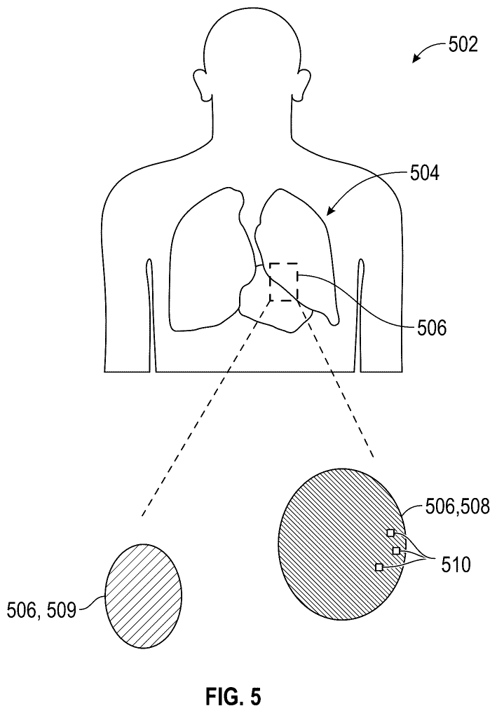

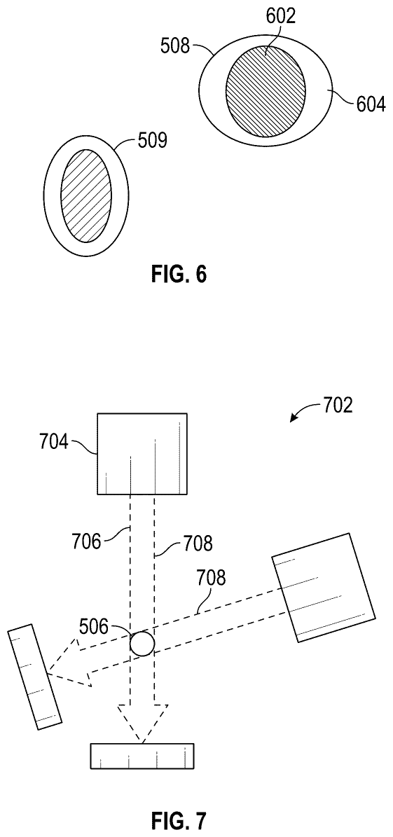

Referring to FIG. 6, voxels of a volumetric image having associated uncertainty values is shown in accordance with embodiments described herein. A visualization of one or more VOIs 506 can be presented as a probability map, where each voxel 510 has an uncertainty value representing whether it is inside or outside of the VOI 506. More particularly, the visualization can include a first probability map corresponding to the target 508 and a second probability map corresponding to the OAR 509. Each of the probability maps can be represented as discrete regions, e.g., a core region 602 and a penumbra region 604, however, it will be appreciated that the entire VOI 506 can include voxels 510 having respective uncertainty values and are not necessarily grouped into particular regions.

The processing device can determine the uncertainty values of the voxels 510 based on parameters associated with the voxels. For example, the processing device may determine and/or assign values for one or more of a shape parameter, an intensity parameter, or an imaging parameter, each of which may be associated with the sources of uncertainty described above. By way of example, the processing device can determine one or more imaging parameters associated with the imaging system used to capture the volumetric image 502, including parameters associated with various artifacts. Values may be assigned to the parameters, and the values may be used to determine the uncertainty values of the voxels 510. More particularly, the uncertainty values of the voxels 510 can be based in part on the one or more shape parameter, intensity parameter, or imaging parameters. Higher uncertainty values may be more likely to occur in voxels 510 within the penumbra region 604, and lower uncertainty values may be more likely to occur in voxels 510 within the core region 602. In an embodiment, the uncertainty values are normalized to have a value within a range of 0 to 1, with voxels 510 having an uncertainty value of 0 being determined to be within the VOI 506 and voxels 510 having an uncertainty value of 1 being determined to not be within the VOI. In an embodiment, the uncertainty values are normalized to have a value withing a range of 0 to 1, with voxels 510 having an uncertainty value less than or equal to a first predetermined value, e.g., 0.5, being determined to be within the VOI 506 and voxels 510 having an uncertainty value of greater than or equal to a second predetermined value (the second predetermined value being the same or different than the first predetermined value), e.g., 0.5 or 0.51, being determined to be within the VOI 506.

The uncertainty values of the voxels 510, which may also be predictions or probabilities of the voxels 510, may be determined using a machine learning method. For the sake of brevity, the applicable machine learning methods are not described in detail, however, it will be appreciated that subsets of machine learning, such as deep learning, may be applied to determine the uncertainty values of the voxels 510, or the predictions or probabilities of the voxels 510, as an output from an input of the volumetric image 502. More particularly, and by way of example, the uncertainty values of the voxels 510 may be determined using a convolutional neural network (CNN), where the input to the CNN is the volumetric image 502, and the output of the CNN is the uncertainty values of the voxels 510, or the predictions or probabilities of the voxels 510. The predictions or probabilities of the voxels 510 may be considered uncertainty values, as used throughout this description, or certainty values. In either case, the value can correspond to a probability that the voxel is inside or outside a VOI. More particularly, the value may be expressed as a value, or an inverse of that value, and be used to make a similar determination, as will be understood by one skilled in the art.

Referring to FIG. 7, a radiation delivery system is shown in accordance with embodiments described herein. At operation 406, the processing device generates beam control data. The beam control data can be used by the apparatus 100 to direct a treatment beam 706 relative to the VOI 506 based on the uncertainty values to meet a predetermined quality metric. For example, the beam control data may be generated to cause the radiation beam to be directed to tissue that is likely to be part of the VOI 506 (when the VOI is the target 508) or to be directed to tissue that is not likely to part of the VOI (when the VOI is the OAR 509). Accordingly, the processing device can generate machine instructions that meet predetermined objectives, e.g., to provide a predetermined dose to the VOI 506.

Radiation therapy treatment “inverse” planning involves solving an optimization problem with an objective function that maximizes dose and dose homogeneity to target certain structures, e.g., the target 508, and to minimize dose to other structures, e.g., the OAR 509. Whereas previous methods of treating such structures would typically involve adding extra margins around the target 508 and, thus, delivering high doses to both tumor and healthy tissue, the method described herein can tailor treatment based on uncertainty to accurately target 508 tumor tissue and reduce dose delivered to healthy tissue.

In an embodiment, the objective is met by setting the predetermined quality metric to be a predetermined minimum dose. For example, the anatomical region 504 can be the target 508, and the predetermined minimum dose may be a dose expected to have a therapeutic effect on the target 508. Dose may be delivered according to beam control data that is determined to meet the predetermined minimum dose criteria. Accordingly, the beam control data can direct the treatment beam 706 to meet the predetermined quality metric of delivering the predetermined minimum dose to the target 508.

The predetermined quality metric can be a predetermined maximum dose. For example, the anatomical region 504 can be the OAR 509, and the predetermined maximum dose may be a dose expected to not adversely affect the OAR function. Dose may be delivered according to the beam control data that is determined to meet the predetermined maximum dose criteria. Accordingly, the beam control data can direct the treatment beam 706 to meet the predetermined quality metric of delivering no more than the predetermined maximum dose to the OAR 509.

The beam control data may be output to a radiation delivery system of the apparatus 100. The radiation delivery system can have a treatment beam generator 704, such as the LINAC described above. The treatment beam generator 704 can generate the treatment beam 706 and direct the treatment beam toward the VOI 506. The beam control data can include values of parameters to control beam delivery. The beam control data can include data to control a beam delivery angle 708 or a MLC configuration (a shape or size of the MLC opening). For example, the treatment beam 706 may be rotated to a position relative to the VOI 506, as shown in FIG. 7, that will cause radiation to meet the predetermined quality metric for treatment of the target 508 and/or avoidance of the OAR 509. Accordingly, the output beam control data can be used by the radiation delivery system to effectively treat the patient based on uncertainty values of the voxels 510 in the volumetric image 502.

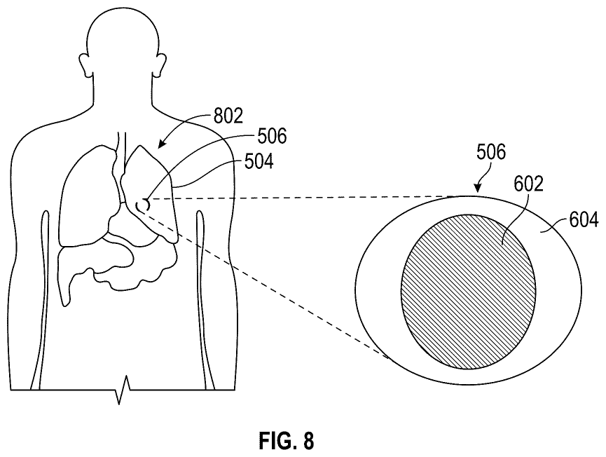

Referring to FIG. 8, a segmentation map representing a volume of interest is shown in accordance with embodiments described herein. The apparatus 100 can generate a segmentation map 802. The segmentation map 802 can be a computer-generated segmentation, e.g., generated using an autosegmentation algorithm, of various anatomies in the patient, including the anatomical region 504 having the VOI 506. For example, the anatomical region 504 can be a lung and the VOI 506 can be a tumor in the lung. The segmentation map 802 can include the several voxels 510 referred to above, which have respective uncertainty values.

In an embodiment, the segmentation map 802 indicates regions of the VOI 506 based on whether the uncertainty values of voxels 510 within the regions are above or below a predetermined threshold. For example, the predetermined threshold can be a value between 0 and 1 indicated the probability that the voxel 510 belongs to the VOI 506. In the illustrated embodiment, the penumbra region 604 can include voxels 510 having uncertainty values above the predetermined threshold, e.g., 0.7, indicating that the displayed penumbra region 604 is less likely to be part of the target 508 that the user seeks to treat. More particularly, the core region 602 can include voxels 510 having uncertainty values below the predetermined threshold, indicating that the displayed penumbra region 604 is more likely to be part of the target 508.

The predetermined threshold may be user-defined, e.g., through a user interface, or may be preset within the operating instructions of the apparatus 100. The use of the predetermined threshold that drives display of a graphical representation of the VOI 506 split into two or more regions based on probability of voxels 510 therein being part of the VOI can be useful in treatment planning. The user may view the segmentation map 802 to gain feedback regarding uncertainty. For example, the penumbra region 604 may be displayed differently than the core region 602, e.g., a different color or blurred, to distinguish areas of higher confidence from areas of lower confidence. The user can gain confidence around whether the segmentation of the anatomies is accurate, and can mark up or modify treatment plans accordingly.

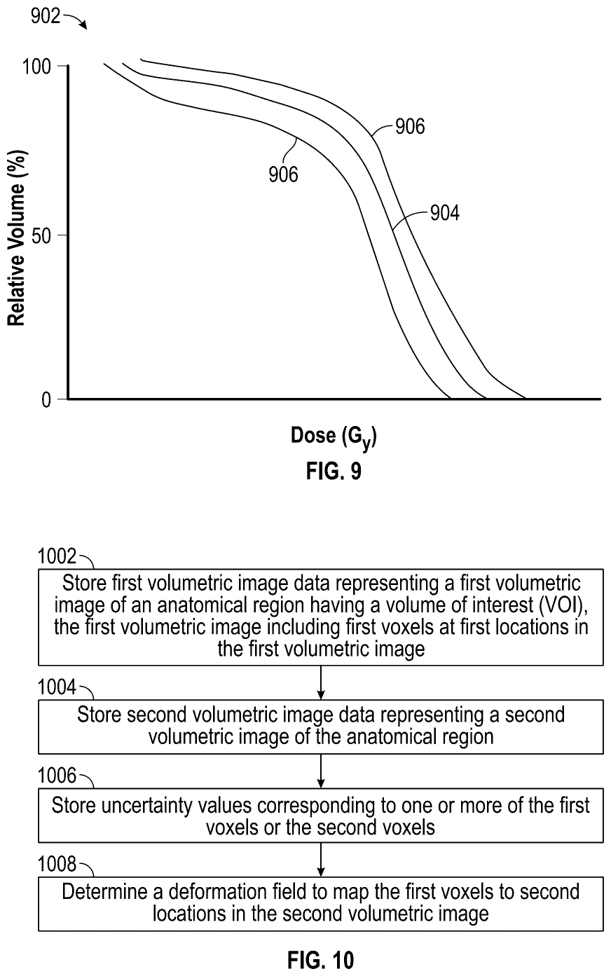

Referring to FIG. 9, a dose-volume histogram for a volume of interest is shown in accordance with embodiments described herein. Treatment plans typically ignore uncertainty associated with the segmented areas of the imaged VOI 506. Constraints to irradiation of the VOI 506, e.g., the target 508 or the OAR 509, usually take the form of DxVx criteria. DxVx criteria can define an acceptable dose per relative volume of the VOI 506. For example, when the VOI 506 is the target 508, the DxVx criteria may define delivery of at most a first predetermined dosage to a predetermined percentage of the target 508 volume. Similarly, when the VOI 506 is the OAR 509, the DxVx criteria may define delivery of at most a second predetermined dosage (which may be less than the first product dosage) to a predetermined percentage of the OAR volume. The dosage may have no confidence level associated with it, however.

In an embodiment, the predetermined quality metric, which the beam control data may be generated to meet, can be associated with a predetermined confidence level. For example, the predetermined quality metric can include DxVx criteria that are associated with a confidence interval. The processing device can generate beam control data that control the treatment beam 706 to realize a treatment plan in which a maximum dosage is delivered to a predetermined percentage of the OAR with a predetermined probability. The predetermined probability can consider the uncertainty of the OAR boundary. For example, the processing device can determine plan quality metrics, which can include DxVx criteria, conformality, or other indices computed as random variables with, for example, a 95% confidence interval. The uncertainty values can therefore drive objectives, e.g., maximizing dose to areas that are more likely in the tumor or minimizing dose to areas that are less likely in the tumor.

The confidence interval associated with the plan quality metric can be represented in a dose-volume histogram (DVH) 902. The DVH 902 can be a histogram relating radiation dose to tissue volume. The DVH 902 may be for the VOI 506 and, thus, can relate radiation dose to the VOI volume. Accordingly, the DVH 902 can include a DVH curve 904 that represents how much dosage each relative volume of the VOI 506 is expected to receive according to the treatment plan.

In an embodiment, the DVH 902 includes a band based on a confidence interval. The DVH 902 for the VOI 506 can be determined by the processing device, based on the volumetric data, and can include the DVH curve 904 between several bounding curves 906 associated with confidence intervals. More particularly, the bounding curves 906 can be based on respective confidence intervals. By way of example, the illustrated bounding curves 906 can include a first bounding curve 906 to the left of the DVH curve 904 and a second bounding curve 906 to the right of the DVH curve 904. The first bounding curve 906 can be associated with a first, e.g., 25%, confidence interval and the second bounding curve 906 can be associated with a second, 95%, confidence interval. The DVH curve 904, between the bounding curves 906, may be associated with a third, 85%, confidence interval, for example. Accordingly, it will be appreciated that the uncertainty values associated with the voxels 510 in the volumetric image 502 can be taken into account when determining the DVH 902, and that the DVH curve(s) 904 can be based on dose to the voxels 510 weighted by the uncertainty values of the voxels 510.

The predetermined quality metric can be selected based on the DVH 902. In an embodiment, DVH curve 904 having several DVH curves (the DVH curve 904 and bounding curves 906) can be presented to a user, e.g., via a display of the apparatus 100. The user may review the curves and make a decision or choice about how to perform a treatment. For example, the user may view the curves and notice that selection of the DVH curve 904 can ensure that there is a high probability that 50% of the VOI 506 will receive the dose between the bounding curves 906 at that level. The user may then select the treatment plan based on that knowledge. Alternatively, the user may choose to be more cautious by selecting a treatment plan associated with the first bounding curve 906. Selection of the treatment plan can cause the processing device to generate the beam control data that maximizes the probability that the treatment objective, e.g., the DxVx criteria, is met.

The DVH curve 904 having the several curves can provide a visualization tool. More particularly, the user can view the DVH curve 904 to validate that the treatment plan will achieve the desired treatment criteria with a particular confidence. Accordingly, the DVH curve 904 can be used both to drive treatment selection and to confirm the user expectation.

As an example of how the method above might impact planning, consider a hypothetical patient with a single target 508 and a single OAR 509. The OAR 509 has uncertainty at its border, but the uncertainty is not homogeneous. That is, the penumbra region 604 of the segmentation map 802 has a thicker tail in some directions than in others. The processing device can generate beam control data that directs the treatment beam 706 around the OAR 509 on the thin-tailed side rather than the thick-tailed side. The approach is similar to using a margin with varying widths on different sides of the VOI 506, but it does not require explicitly drawing the margins by a user, because the beam control data is derived directly from the uncertainty in the segmentation map 802.

The above description focuses primarily on treatment planning, however, it will be appreciated that uncertainty may similarly be leveraged to generate beam control data during treatment. More particularly, radiation can be delivered to organs tracked in real-time with uncertain boundaries.

Real-time imaging allows for tracking of OARs and targets 508 during treatment and subsequent modification of a treatment plan. For example, based on organ tracking, a jaw or MLC adjustment can be made. Similarly, beam control data may be modified to account for VOI movements to hit the target 508 and spare the OAR 509.

Accurate delineation of a three-dimensional VOI boundary in real-time is technically challenging, so existing tracking systems may rely on assumptions about motion during treatment. For example, the tracking systems might assume that VOI motion has only a translation component (no rotation or deformation) or that the motion of all VOIs is correlated and in-phase. Furthermore, the dose delivered to the OARs may only be estimated during planning and not updated during treatment.

In an embodiment, a tracking algorithm that explicitly models uncertainty in the contour boundaries of a VOI and uncertainty in the dose constraints of the VOI, and updates both the boundary uncertainty and the delivered dose during treatment delivery, is contemplated. For example, the apparatus 100 can use a combined weighting of uncertainty and dose constraints to guide the tracking during treatment.

It is contemplated that the apparatus 100 can begin treatment with the segmentation map 802, which incorporates uncertainty, as described above. Each voxel 510 in the uncertainty map can contain a probability that the voxel 510 belongs to one or more VOIs 506 in the segmentation map 802. During treatment, the apparatus 100 can generate motion data or image data, and the segmentation map 802 can be updated based on such data. For example, laser sensors may limit uncertainty stemming from a respiratory phase, and 2D x-ray snapshots may reduce uncertainty of VOI 506 boundaries within an imaging plane. By contrast, in the absence of motion data input, the boundary uncertainty may actually increase. Accordingly, the respective uncertainty values of the several voxels 510 may change.

Referring back to operation 404, in the context of real-time organ tracking, the determining of the several voxels 510 can include determining that the respective uncertainty values have changed from prior uncertainty values. More particularly, the uncertainty values may differ from prior uncertainty values determined in a treatment planning stage. Accordingly, the method illustrated in FIG. 4 may apply to either a planning stage or a treatment stage.

When the method is applied to the treatment stage, generating the beam control data at operation 406 can include modifying prior beam control data to meet the predetermined quality metric. For example, the beam control data can be changed, relative to beam control data generated in the planning stage, such that the probability of meeting the DxVx criteria during the planned fraction is met.

As an example to compare the method of FIG. 4, as applied to the treatment stage, to existing methods, consider a hypothetical patient with a single target 508 and a single OAR that is highly radiosensitive and critical. An existing tracking system may estimate zero translation from a reference position of the OAR, and may proceed with treatment as planned. By comparison, the method using uncertainty may recognize that volumetric images 502 captured during treatment have blurred borders around the OAR, indicating uncertainty of those regions with respect to whether those regions are part of the OAR. The apparatus 100 may accordingly alter the beam control data to not pass through the penumbra region 604 and to weight the treatment beam 706 according to dose constraints to be more conservative until uncertainty is reduced, thus ensuring that a probability of exceeding Dx Vx limitations to the OAR is low. Similarly, if the penumbra region 604 to the target VOI 506 broadens, more dose can be delivered to the penumbra region 604 than originally planned in order to ensure target coverage with a high degree of probability.

As described above, uncertainty can be integrated into pre-treatment planning and real-time organ tracking methods. Uncertainty can also be useful in deformable image registration and the accumulation of delivered dose, as described below.

### Image Registration Using Uncertainty

In online adaptive radiotherapy (OART), an image of a patient taken on a day of treatment may be used to adapt a radiation therapy plan based on changes in anatomy since the acquisition of an original planning image. High-quality plan adaptation requires highly accurate deformation fields to map voxels 510 between daily images, taken on the day of treatment, and the planning image. As described below, a method includes using a probability map version of the VOI 506 to compute the highly accurate deformation field.

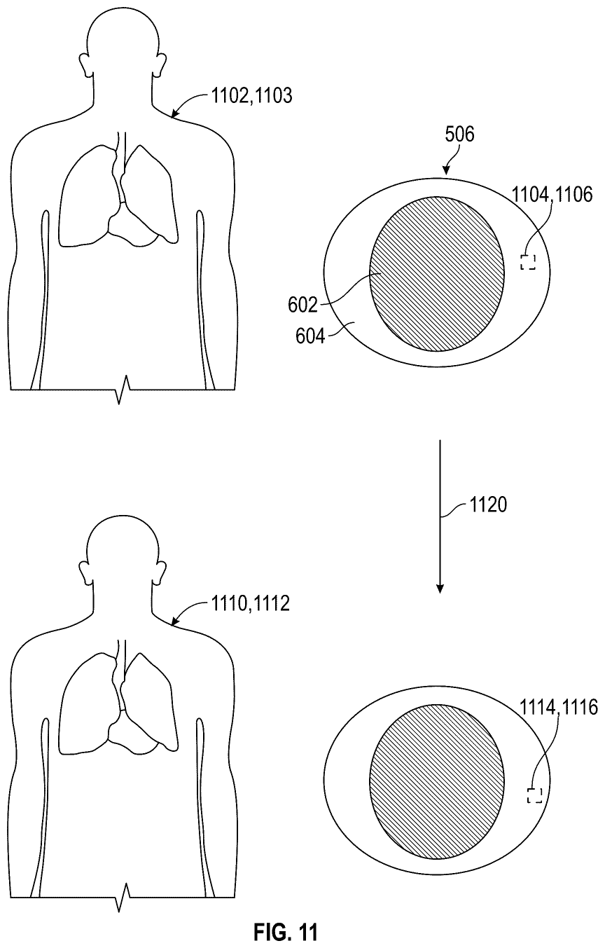

Referring to FIG. 10, a flow diagram of a method of determining a deformation field is shown in accordance with embodiments described herein. The method illustrated in FIG. 10 can be understood with reference to FIGS. 11-13 and, thus, FIGS. 10-13 are alternately referred to below.

Referring to FIG. 11, volumetric images mapped to each other by a deformation field is shown in accordance with embodiments described herein. At operation 1002, a memory of the apparatus 100 stores first volumetric image data representing a first volumetric image 1102 of the anatomical region 504 having the VOI 506. The first volumetric image 1102 can include first voxels 1104 that, as described above, correspond to first uncertainty values. The uncertainty values can be determined by the processing device and included in the first volumetric image data.

The first voxels 1104 can be at respective first locations 1106 in the first volumetric image 1102. The first uncertainty values can represent probabilities that the corresponding first voxels 1104 are part of the VOI 506. For example, a first voxel 1104 may be located at a first location 1106 in the penumbra region 604 of the VOI 506. Accordingly, the first voxel 1104 may be associated with a relatively low probability of being part of the VOI 506. Alternatively, first voxels 1104 located in the core region 602 can have a relatively high probability of being part of the VOI 506.

At operation 1004, second volumetric image data is stored by the memory. The second volumetric image data represents a second volumetric image 1110 of the anatomical region 504. The second volumetric image 1110 can include second voxels 1114. Like the first voxels 1104 of the first volumetric image 1102, the second voxels 1114 of the second volumetric image 1110 can have respective second locations 1116 in the second volumetric image 1110. The second volumetric image data may also include second uncertainty values corresponding to the second voxels 1114. The second uncertainty values can represent probabilities that the corresponding second voxels 1114 are part of the VOI 506. For example, a second voxel 1114 may be located at a second location 1116 in the penumbra region 604 of the VOI 506. Accordingly, the second voxel 1114 may be associated with a relatively low probability of being part of the VOI 506. Alternatively, second voxels 1114 located in the core region 602 can have a relatively high probability of being part of the VOI 506.

At operation 1006, the uncertainty values may be stored. For example, the uncertainty values corresponding to one or more of the first voxels 1104 or the second voxels 1114 may be stored by the memory. The stored uncertainty values may, therefore, include the first uncertainty values and the second uncertainty values.

At operation 1008, the processing device, which is operatively coupled to the memory, determines a deformation field 1120. The deformation field 1120 maps the first voxels 1104 of the first volumetric image 1102 to the second locations 1116 in the second volumetric image 1110. More particularly, the first voxels 1104 can be mapped to the second voxels 1114 of the second volumetric image 1110.

The deformable image registration contemplated herein considers VOIs probabilistically, where each voxel in the volumetric image(s) corresponds to a probability (or confidence level) that the voxel belongs to each VOI. More particularly, the deformation field 1120, which is determined by the processing device, can be based in part on the uncertainty values. Here and throughout the description, the term “based in part on” or “based on” can be equivalent to being influenced by or informed by the uncertainty values. The deformation may not, however, be solely based on the uncertainty values. The deformation can be based on several factors, as indicated by the equation below. For example, the deformation field 1120 may account for image intensity, estimated VOI locations, etc., and the deformation field 1120 may include uncertainty as one factor, e.g., a primary factor or a secondary factor.

In an embodiment, the deformation can find a solution in which locations in the first volumetric image 1102 that have a high probability of being within the VOI 506 are mapped to locations in the second volumetric image 1110 that have a similarly high probability of being within the VOI 506. Similarly, locations in the first volumetric image 1102 that have a low probability of being within the VOI 506 can be mapped to locations in the second volumetric image 1110 that have a similarly low probability of being within the VOI 506. The deformation can be computed using an optimization procedure to solve the following equation:

Φ=argmax(Si(f,s°Φ)+λ₁Ov(f_{c},s_{c}°Φ)+λ₂R(Φ))

In the above equation, the term Φ is the deformation field 1120. The deformation field Φ 1120 is determined by solving the equation to maximize the value of the term inside argmax( ).

Certain terms refer to the volumetric images and their respective VOI maps. The term f refers to the first volumetric image 1102. The term s refers to the second volumetric image 1110. The term f_{c} refers to the VOI map of the first volumetric image 1102. The term s_{c} refers to the VOI map of the second volumetric image 1110.

The term Si is an intensity-based image similarity function. The term can allow deformation to be performed based in part on matching voxels 510 having similar intensities in both images. More particularly, the term can be maximized between the two images by deforming the first volumetric image 1102 to cause voxels 510 of the first volumetric image 1102 to most closely match intensities of the second voxels 1114 in the second volumetric image 1110.

The term Ov is a VOI overlap function. The overlap function can seek to match two VOIs as closely as possible.

The term R is a regularization function. For example, the regularization term can be maximized to make the deformation of the tissue as smooth as possible.

The terms λ₁ and λ₂ are weighting factors. Weighting factors can be used to weight intensity more or less. For example, a larger value of the weighting factor λ₁ may cause VOI overlap to be more important, and a larger value of the weighting factor λ₂ may cause deformation smoothness to be more important in some areas (e.g., in regions having lower probability of being part of the VOI) than other areas.

The above equation, unlike existing deformable image registration algorithms, takes the uncertainty values of the voxels 510 into account. For example, the terms f_{c} and s_{c} can refer to probability maps rather than binary masks. Furthermore, the term Ov can include a probabilistic overlap function such as a sum of square differences in probability or cross correlation. Accordingly, based on the uncertainty values, voxels 510 with higher probability of being in the VOI 506 in the first volumetric image 1102 are more strongly pushed towards overlapping with the VOI in the second volumetric image 1110 than those with a lower probability. Likewise, voxels 510 with a lower probability of being in the VOI 506 in the first volumetric image 1102 are more strongly pushed towards a non-overlapping position with the VOI in the second volumetric image 1110. This allows the optimization algorithm to be influenced by VOI inputs without following erroneous contour labels in subjective regions at the edges of the VOIs to lead the deformation field astray.

In an embodiment, the deformation is used to register a planning image 1103 taken during a planning phase to a daily image 1112 taken during a treatment phase. The first volumetric image 1102 may be a planning image 1103 of the VOI 506, and the second volumetric image 1110 can be a daily image 1112 of the VOI 506. Accordingly, the second volumetric image 1110 can be captured after the planning image 1103.

In an embodiment, the deformation is used to register the daily image 1112 to the planning image 1103. The first volumetric image 1102 can include the daily image 1112 of the VOI 506, and the second volumetric image 1110 can include the planning image 1103 of the VOI. Accordingly, the second volumetric image 1110 can be captured before the first volumetric image 1102.

As described above, the deformation field 1120 can register a planning image 1103 to a daily image 1112 and vice versa. The registration may be based on uncertainty values associated with the image voxels 510. More particularly, one or more of the first volumetric image 1102 or the second volumetric image 1110 may have uncertainty values corresponding to respective voxels 510, and the deformation field 1120 can be based in part on such voxels 510. Accordingly, the deformation field 1120 may be based in part on one or more of first uncertainty values corresponding to the first voxels 1104 in the first volumetric image 1102 or second uncertainty values corresponding to the second voxels 1114 in the second volumetric image 1110. The deformation field 1120 can use the uncertainty values to map all of the voxels 510 in a three-dimensional volume enclosing the first voxels 1104 to locations in another three-dimensional volume enclosing the second voxels 1114. The first voxels 1104 are not explicitly mapped to the second voxels 1114, but rather, the first voxels 1104 are mapped to new locations at which the second voxels 1114 may be located. Accordingly, the deformation field 1120 maps the VOIs to each other.

In a first mode, the registration algorithm can use probabilistic VOI maps (f_{c} and s_{c}) from both the first volumetric image 1102 and the second volumetric image 1110. More particularly, the computation of the deformation field 1120 can be based in part on the first voxel uncertainty values of the first VOI map and on the second voxel uncertainty values of the second VOI map. That is, the computation can use the uncertainty of the first and second VOI maps and not only uncertainty from one of the VOI maps. In such case, unthresholded VOI probability maps from each of the first volumetric image 1102 and the second volumetric image 1110 can be used as inputs into the image registration optimization algorithm.

In a second mode, an image having hard segmentation may be mapped to an image having a probabilistic segmentation. More particularly, uncertainty values may be associated with voxels 510 of only one of each of the images. The registration algorithm can use a binary VOI map from the first volumetric image 1102 (f_{c}) and a probabilistic VOI map from the second volumetric image 1110 (s_{c}). The binary VOI map can be thresholded by a user by determining the VOI map based on whether the uncertainty values are above a predetermined value. By contrast, the probabilistic VOI map can retain raw uncertainty values for the VOI map that is used in the deformation field 1120 calculation to guide the registration algorithm.

Above, the modes are described in reference to first and second volumetric images, and it will be appreciated that the modes may be applied to compute deformation between images that are taken before or after each other. More particularly, the mapping is invertible, e.g., planning images 1103 may be mapped to daily images 1112 or daily images 1112 may be mapped to planning images 1103. Accordingly, mapping may refer to the images being mapped to each other, not in any particular time-based manner.

The two-way mapping can allow the image registration to be used for different purposes. For example, the image registration can be used to perform treatment beam control in an anatomy that has moved between the planning image 1103 and the daily image 1112. Furthermore, as discussed below, the approach can allow users to accumulate dose from multiple fractions more accurately, and thus, to better adapt treatment plans as the delivered dose begins to diverge from a directive of a physician, e.g., due to a change in the anatomy. Advantageously, in any of these applications, the deformation can be computed between images in a manner that is robust to small segmentation errors. More particularly, rather than driving deformation to force surfaces from each of the volumetric images to match, deformation can be driven by uncertainty values that encourages matching probabilistically. Organ structures in the two images that have a higher probability of corresponding to each other are more likely to overlap after deformation than those with lower probability. This can result in less deformation errors and achieve more accurate contours and deformation fields 1120.

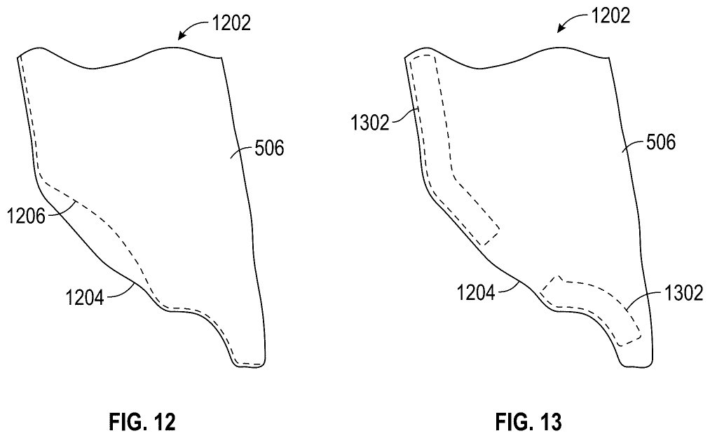

Referring to FIG. 12, a deformed image having a contour is shown in accordance with embodiments described herein. Uncertainty in contours of an image, such as the planning image 1103, can be propagated to uncertainty within the deformation. In an embodiment, the processing device generates a deformed image 1202 representing the VOI 506, e.g., a lung. The deformed image 1202 can be generated based on the deformation of the first volumetric image 1102. For example, the deformation field 1120 can be applied using the probabilistic inputs of the planning and daily images 1112 to produce the deformed image 1202. As shown, the deformed image 1202 can include a contour 1204 of the VOI 506.

A user may modify the contour 1204. For example, the apparatus 100 may determine that a portion of the contour 1204 corresponds to voxels 510 having uncertainty values within a particular range, e.g., indicating an increased likelihood that the voxels 510 may actually not be part of the VOI 506. The processing device can generate an alert indicating such determination. For example, the portion of the contour 1204 may have a predetermined color, e.g., red, in a displayed segmentation. Alternatively, the user may view the contour 1204 and, based on experience, may decide to adjust the contour line. In any case, the processing device can receive a user input modifying the contour 1204 of the VOI 506.

The user input can change the contour 1204 to a modified contour 1206 1204. The modified contour 1206 can include a change to the portion of the second volumetric image 1110 that is considered to be part of the VOI 506. For example, the user can select an anchor point of the contour 1204 and drag it to change the contour shape, which can alter an area circumscribed by the contour.

The processing device may, in response to the user input modifying the contour 1204, determine a second deformation field 1120. The second deformation field 1120 can be based in part on the modified contour 1206. Like the first deformation field 1120 used to generate the contour 1204, use the above equation can map voxels 510 of the first volumetric image 1102 to locations in the second volumetric image 1110. The locations, however, may be different. Accordingly, rather than being mapped to second locations 1116 in the deformed image 1202, the voxels 510 can be mapped to third locations in the second volumetric image 1110. The re-mapping of the voxels 510 to the second volumetric image 1110 may drive the generation of a modified, deformed image 1202. The modified, deformed image 1202 can have the modified contour 1206 and can include voxels 510 determined to be part of the VOI 506 in the second volumetric image 1110. Accordingly, upon editing the deformed contours 1204 at the uncertain areas of the treatment image, additional optimization can be performed to incorporate the extra information of the modified contour 1206, and propagate those edits to the full deformation field 1120.

Referring to FIG. 13, a deformed imaging having an uncertain region is shown in accordance with embodiments described herein. As described above, the deformed image 1202 may be generated to include the contour 1204 of the VOI 506. In an embodiment, the deformed image 1202 indicates an uncertain region 1302 of the contour 1204. The uncertain region 1302 may be one mode of generating an alert to the user regarding uncertainty associated with the deformed image 1202. For example, the uncertain region 1302 can be a region of the image that is surrounding by a dotted line, colored differently than the remainder of the VOI, etc. The visual representation of the uncertain region 1302 can indicate to the user that the portion of the VOI 506 in the image has a lower probability of being part of the VOI than, for example, another portion of the VOI not similarly rendered. The user may provide the user input, as described above, to modify the uncertain region of the contour 1204 of the VOI 506. For example, the user may drag lines indicating a perimeter of the uncertain region to re-size the region. Accordingly, the second deformation field 1120 can be determined and used to map the first volumetric image 1102 to the second volumetric image 1110 having the altered geometry.

According to the above process, a user can alter segmentation and the alteration can be fed back into the optimization of the deformation. The optimization, accordingly, gets updated based on user feedback related to the initial deformation. The deformation map can therefore be improved indirectly by updating the contours of the deformed image 1202. Several iterations may be performed, with revisions being made at each iteration, to update and optimize the deformation field 1120.

### Dose Accumulation Using Uncertainty

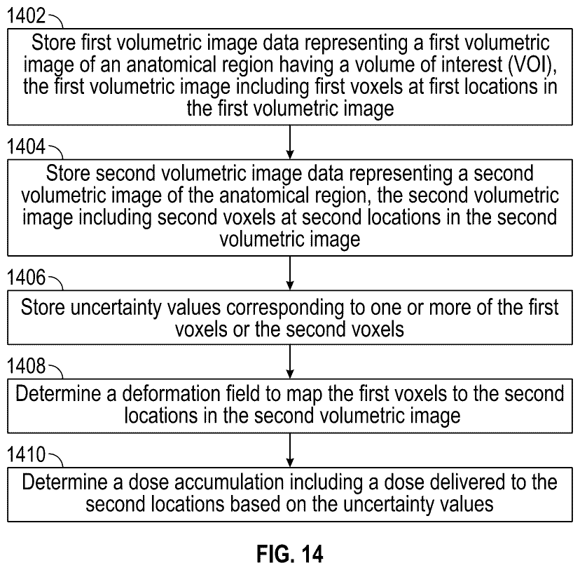

Referring to FIG. 14, a flow diagram of a method of determining a dose accumulation based on uncertainty values is shown in accordance with embodiments described herein. An application of the uncertainty-influenced deformation is dose accumulation. More particularly, the processing device can determine a dose accumulation including a dose delivered to the second locations 1116 in the second volumetric image 1110. The total dose over a course of treatment to a voxel 510 can be computed as a sum of the per-fraction doses to that voxel 510 weighted by confidence in the mapping between that voxel 510 on the first volumetric image 1102 (e.g., the planning image 1103) and its corresponding voxel 510 on the second volumetric image 1110 (e.g., the daily image 1112).

At operation 1402, first volumetric image data representing the first volumetric image 1102 of the anatomic region having the VOI 506 is stored by memory of the apparatus 100. As described above, the first volumetric image 1102 includes the first voxels 1104 at first locations 1106 in the first volumetric image 1102.

At operation 1404, second volumetric image data representing the second volumetric image 1110 of the anatomical region 504 is stored by the memory. The second volumetric image 1110 includes second voxels 1114 at second locations 1116 in the second volumetric image 1110. The memory may also store, at operation 1406, uncertainty values corresponding to one or more of the first voxels 1104 or the second voxels 1114. As described above, the deformation field 1120 can be applied to map the first volumetric image 1102 to the second volumetric image 1110. More particularly, at operation 1408, deformation field 1120 may be determined to map the first voxels 1104 to the second locations 1116 in the second volumetric image 1110.

The deformation may be used to map doses applied to voxels 510 in one or more of the volumetric images 502. More particularly, doses applied to the first locations 1106 in the first volumetric image 1102 may be summed with doses applied to the second locations 1116 in the second volumetric image 1110 to determine dose accumulation to the voxels 510 at those locations. The dose accumulation can therefore be based on the uncertainty values. More particularly, at operation 1410, the dose accumulation can be determined, and the dose accumulation can include a dose delivered to the second locations 1116 based on the uncertainty values.

Dose accumulation, as performed at operation 1410, can be determined based on uncertainty values of one or more of the first voxels 1104 or the second voxels 1114. For example, the dose accumulation can be determined based on the uncertainty values corresponding to the first voxels 1104 at the first locations 1106. Alternatively or additionally, the dose accumulation can be determined based on the uncertainty values corresponding to the second voxels 1114 at the second locations 1116. The uncertainty values corresponding to the voxels 510 that are being summed, to determine dose accumulation, can be associated with the dosage values. For example, dosage applied to the first voxels 1104 at the first locations 1106 can be associated with the uncertainty values, and that uncertainty may be included in a representation for the user to observe. The user may view a dose accumulation to the VOI 506 which may include, for example, various dosage curves indicating that the VOI has received a certain dosage with a given confidence level. The confidence level can change over different fractions, as the voxels 510 that are mapped to the VOI 506 are more or less certain to be part of the VOI. Accordingly, the uncertainty values associated with voxels 510 can be used to present confidence that a particular treatment objective, such as dose accumulation or dose distribution, has been achieved.

Referring to FIG. 15, a block diagram of an example computing device that may perform one or more of the operations described herein is shown in accordance with embodiments described herein. Computing device 1500 may be connected to other computing devices in a LAN, an intranet, an extranet, and/or the Internet. The computing device may operate in the capacity of a server machine in client-server network environment or in the capacity of a client in a peer-to-peer network environment. The computing device may be provided by a personal computer (PC), a set-top box (STB), a server, a network router, switch or bridge, or any machine capable of executing a set of instructions (sequential or otherwise) that specify actions to be taken by that machine. Further, while only a single computing device is illustrated, the term “computing device” shall also be taken to include any collection of computing devices that individually or jointly execute a set (or multiple sets) of instructions to perform the methods discussed herein.

The example computing device 1500 may include a processing device (e.g., a general purpose processor, a PLD, etc.) 1502, a main memory 1504 (e.g., synchronous dynamic random access memory (DRAM), read-only memory (ROM)), a static memory 1506 (e.g., flash memory and a data storage device 1518), which may communicate with each other via a bus 1530.

Processing device 1502 may be provided by one or more general-purpose processing devices such as a microprocessor, central processing unit, or the like. In an illustrative example, processing device 1502 may comprise a complex instruction set computing (CISC) microprocessor, reduced instruction set computing (RISC) microprocessor, very long instruction word (VLIW) microprocessor, or a processor implementing other instruction sets or processors implementing a combination of instruction sets. Processing device 1502 may also comprise one or more special-purpose processing devices such as an application specific integrated circuit (ASIC), a field programmable gate array (FPGA), a digital signal processor (DSP), network processor, or the like. The processing device 1502 may be configured to execute the operations described herein, in accordance with one or more aspects of the present disclosure, for performing the operations and steps discussed herein. For example, the imaging uncertainty instructions 1525 may include instructions for determining voxels having respective uncertainty values or generating beam control data based on the uncertainty values.

Computing device 1500 may further include a network interface device 1508 which may communicate with a network 1520. The computing device 1500 also may include a video display unit 1510 (e.g., a liquid crystal display (LCD) or a cathode ray tube (CRT)), an alphanumeric input device 1512 (e.g., a keyboard), a cursor control device 1514 (e.g., a mouse) and an acoustic signal generation device 1516 (e.g., a speaker). In one embodiment, video display unit 1510, alphanumeric input device 1512, and cursor control device 1514 may be combined into a single component or device (e.g., an LCD touch screen).

Data storage device 1518 may include a computer-readable storage medium 1528 on which may be stored one or more sets of instructions that may include imaging uncertainty instructions 1525 for carrying out the operations described herein, in accordance with one or more aspects of the present disclosure. The instructions may also reside, completely or at least partially, within main memory 1504 and/or within processing device 1502 during execution thereof by computing device 1500, main memory 1504 and processing device 1502 also constituting computer-readable media. The instructions may further be transmitted or received over a network 1520 via network interface device 1508.

While computer-readable storage medium 1528 is shown in an illustrative example to be a single medium, the term “computer-readable storage medium” should be taken to include a single medium or multiple media (e.g., a centralized or distributed database and/or associated caches and servers) that store the one or more sets of instructions. The term “computer-readable storage medium” shall also be taken to include any medium that is capable of storing, encoding or carrying a set of instructions for execution by the machine and that cause the machine to perform the methods described herein. The term “computer-readable storage medium” shall accordingly be taken to include, but not be limited to, solid-state memories, optical media and magnetic media.

It should be noted that the methods and apparatus described herein are not limited to use only with medical diagnostic imaging and treatment. In alternative implementations, the methods and apparatus herein may be used in applications outside of the medical technology field, such as industrial imaging and non-destructive testing of materials. In such applications, for example, “treatment” may refer generally to the effectuation of an operation controlled by the treatment planning system, such as the application of a beam (e.g., radiation, acoustic, etc.) and “target” may refer to a non-anatomical object or area.

The preceding description sets forth numerous specific details such as examples of specific systems, components, methods, and so forth, in order to provide a good understanding of several embodiments of the present disclosure. It will be apparent to one skilled in the art, however, that at least some embodiments of the present disclosure may be practiced without these specific details. In other instances, well-known components or methods are not described in detail or are presented in simple block diagram format in order to avoid unnecessarily obscuring the present disclosure. Thus, the specific details set forth are merely exemplary. Particular embodiments may vary from these exemplary details and still be contemplated to be within the scope of the present disclosure.

Reference throughout this specification to “one embodiment” or “an embodiment” means that a particular feature, structure, or characteristic described in connection with the embodiments included in at least one embodiment. Thus, the appearances of the phrase “in one embodiment” or “in an embodiment” in various places throughout this specification are not necessarily all referring to the same embodiment.

Although the operations of the methods herein are shown and described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent or alternating manner.

The above description of illustrated implementations of the invention, including what is described in the Abstract, is not intended to be exhaustive or to limit the invention to the precise forms disclosed. While specific implementations of, and examples for, the invention are described herein for illustrative purposes, various equivalent modifications are possible within the scope of the invention, as those skilled in the relevant art will recognize. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an implementation” or “one implementation” throughout is not intended to mean the same embodiment or implementation unless described as such. Furthermore, the terms “first,” “second,” “third,” “fourth,” etc. as used herein are meant as labels to distinguish among different elements and may not necessarily have an ordinal meaning according to their numerical designation.

What is claimed is:

1. An apparatus, comprising:
a memory to:
store first volumetric image data representing a first volumetric image of an anatomical region having a volume of interest (VOI), wherein the first volumetric image includes a plurality of first voxels at first locations in the first volumetric image, wherein the first volumetric image data includes first uncertainty values corresponding to the plurality of first voxels, and wherein the first uncertainty values represent probabilities that the corresponding plurality of first voxels are part of the VOI, and
store second volumetric image data representing a second volumetric image of the anatomical region; and
a processing device operatively coupled to the memory to determine a deformation field to map the plurality of first voxels to second locations in the second volumetric image, wherein the deformation field is based in part on the first uncertainty values.

2. The apparatus of claim 1, wherein the second volumetric image includes a plurality of second voxels, wherein the second volumetric image data includes second uncertainty values corresponding to the plurality of second voxels, wherein the second uncertainty values represent probabilities that the corresponding plurality of second voxels are part of the VOI, and wherein the deformation field is based in part on the second uncertainty values.

3. The apparatus of claim 1, wherein the first volumetric image includes a daily image of the VOI, and wherein the second volumetric image includes a planning image of the VOI captured before the daily image.

4. The apparatus of claim 1, wherein the first volumetric image includes a planning image of the VOI, and wherein the second volumetric image includes a daily image of the VOI captured after the planning image.

5. The apparatus of claim 1, wherein the processing device is further to:
generate a deformed image representing the VOI, wherein the deformed image includes a contour of the VOI;
receive a user input modifying the contour of the VOI; and
determine a second deformation field to map the plurality of first voxels of the first volumetric image to third locations in the second volumetric image, wherein the deformation field is based in part on the modified contour.

6. The apparatus of claim 1, wherein the processing device is further to:
generate a deformed image representing the VOI, wherein the deformed image includes a contour of the VOI, and wherein the deformed image indicates an uncertain region of the contour; and
receive a user input modifying the uncertain region of the contour of the VOI.

7. The apparatus of claim 1, wherein the processing device is further to determine a dose accumulation including a dose delivered to the second locations.

8. A method, comprising:
store, by a memory of an apparatus, first volumetric image data representing a first volumetric image of an anatomical region having a volume of interest (VOI), wherein the first volumetric image includes a plurality of first voxels at first locations in the first volumetric image, wherein the first volumetric image data includes first uncertainty values corresponding to the plurality of first voxels, and wherein the first uncertainty values represent probabilities that the corresponding plurality of first voxels are part of the VOI;
store, by the memory, second volumetric image data representing a second volumetric image of the anatomical region; and
determine, by a processing device of the apparatus, a deformation field to map the plurality of first voxels to second locations in the second volumetric image, wherein the deformation field is based in part on the first uncertainty values.

9. The method of claim 8, wherein the second volumetric image includes a plurality of second voxels, wherein the second volumetric image data includes second uncertainty values corresponding to the plurality of second voxels, wherein the second uncertainty values represent probabilities that the corresponding plurality of second voxels are part of the VOI, and wherein the deformation field is based in part on the second uncertainty values.

10. The method of claim 8, wherein the first volumetric image includes a daily image of the VOI, and wherein the second volumetric image includes a planning image of the VOI captured before the daily image.

11. The method of claim 8, wherein the first volumetric image includes a planning image of the VOI, and wherein the second volumetric image includes a daily image of the VOI captured after the planning image.

12. The method of claim 8 further comprising:
generating, by the processing device, a deformed image representing the VOI, wherein the deformed image includes a contour of the VOI;
receiving, by the processing device, a user input modifying the contour of the VOI; and
determining, by the processing device, a second deformation field to map the plurality of first voxels of the first volumetric image to third locations in the second volumetric image, wherein the deformation field is based in part on the modified contour.

13. The method of claim 8 further comprising:
generating, by the processing device, a deformed image representing the VOI, wherein the deformed image includes a contour of the VOI, and wherein the deformed image indicates an uncertain region of the contour; and
receiving, by the processing device, a user input modifying the uncertain region of the contour of the VOI.

14. The method of claim 8 further comprising determining, by the processing device, a dose accumulation including a dose delivered to the second locations.

15. A non-transitory computer-readable storage medium including instructions which, when executed by a processing device of an apparatus, cause the apparatus to:
store first volumetric image data representing a first volumetric image of an anatomical region having a volume of interest (VOI), wherein the first volumetric image includes a plurality of first voxels at first locations in the first volumetric image, wherein the first volumetric image data includes first uncertainty values corresponding to the plurality of first voxels, and wherein the first uncertainty values represent probabilities that the corresponding plurality of first voxels are part of the VOI;
store second volumetric image data representing a second volumetric image of the anatomical region; and
determine a deformation field to map the plurality of first voxels to second locations in the second volumetric image, wherein the deformation field is based in part on the first uncertainty values.

16. The non-transitory computer-readable storage medium of claim 15, wherein the second volumetric image includes a plurality of second voxels, wherein the second volumetric image data includes second uncertainty values corresponding to the plurality of second voxels, wherein the second uncertainty values represent probabilities that the corresponding plurality of second voxels are part of the VOI, and wherein the deformation field is based in part on the second uncertainty values.

17. The non-transitory computer-readable storage medium of claim 15, wherein the first volumetric image includes a daily image of the VOI, and wherein the second volumetric image includes a planning image of the VOI captured before the daily image.

18. The non-transitory computer-readable storage medium of claim 15, wherein the first volumetric image includes a planning image of the VOI, and wherein the second volumetric image includes a daily image of the VOI captured after the planning image.

19. The non-transitory computer-readable storage medium of claim 15 further causing the apparatus to:
generate a deformed image representing the VOI, wherein the deformed image includes a contour of the VOI;
receive a user input modifying the contour of the VOI; and
determine a second deformation field to map the plurality of first voxels of the first volumetric image to third locations in the second volumetric image, wherein the deformation field is based in part on the modified contour.

20. The non-transitory computer-readable storage medium of claim 15 further causing the apparatus to:
generate a deformed image representing the VOI, wherein the deformed image includes a contour of the VOI, and wherein the deformed image indicates an uncertain region of the contour; and
receive a user input modifying the uncertain region of the contour of the VOI.

21. The non-transitory computer-readable storage medium of claim 15 further causing the apparatus to determine a dose accumulation including a dose delivered to the second locations.

22. An apparatus, comprising:
a memory to:
store first volumetric image data representing a first volumetric image of an anatomical region having a volume of interest (VOI), wherein the first volumetric image includes a plurality of first voxels at first locations in the first volumetric image,
store second volumetric image data representing a second volumetric image of the anatomical region, wherein the second volumetric image includes a plurality of second voxels at second locations in the second volumetric image, and
storing uncertainty values corresponding to one or more of the plurality of first voxels or the plurality of second voxels, and wherein the uncertainty values represent probabilities that the corresponding plurality of voxels are part of the VOI; and
a processing device operatively coupled to the memory to:
map the plurality of first voxels to the second locations in the second volumetric image, and
determine a dose accumulation including a dose delivered to the second locations based on the uncertainty values.

23. The apparatus of claim 22, wherein the dose accumulation is determined based on the uncertainty values corresponding to the plurality of first voxels.

24. The apparatus of claim 22, wherein the dose accumulation is determined based on the uncertainty values corresponding to the plurality of second voxels.

25. A method, comprising:
storing, by a memory of an apparatus, first volumetric image data representing a first volumetric image of an anatomical region having a volume of interest (VOI), wherein the first volumetric image includes a plurality of first voxels at first locations in the first volumetric image;
storing, by the memory, second volumetric image data representing a second volumetric image of the anatomical region, wherein the second volumetric image includes a plurality of second voxels at second locations in the second volumetric image;
storing uncertainty values corresponding to one or more of the plurality of first voxels or the plurality of second voxels, and wherein the uncertainty values represent probabilities that the corresponding plurality of voxels are part of the VOI;
mapping, by a processing device of the apparatus, the plurality of first voxels to the second locations in the second volumetric image; and
determining, by the processing device, a dose accumulation including a dose delivered to the second locations based on the uncertainty values.

26. A non-transitory computer-readable storage medium including instructions which, when executed by a processing device of an apparatus, cause the apparatus to:
store first volumetric image data representing a first volumetric image of an anatomical region having a volume of interest (VOI), wherein the first volumetric image includes a plurality of first voxels at first locations in the first volumetric image;
store second volumetric image data representing a second volumetric image of the anatomical region, wherein the second volumetric image includes a plurality of second voxels at second locations in the second volumetric image;
store uncertainty values corresponding to one or more of the plurality of first voxels or the plurality of second voxels, and wherein the uncertainty values represent probabilities that the corresponding plurality of voxels are part of the VOI;
map the plurality of first voxels to the second locations in the second volumetric image; and
determine a dose accumulation including a dose delivered to the second locations based on the uncertainty values.
